(19) United States
(12) Patent Application Publication
(10) Pub. No.: US 2006/0100562 A1
(43) Pub. Date: May 11, 2006

(54) Hip distraction system

(76) Inventor: James Christopher Pamplin, Plymouth, NH (US)

Correspondence Address: James Pamplin, 79 Highland St., Plymouth, NH, 03264, US

(21) Appl. No.: 10/984,140

(22) Filed: Nov. 9, 2004

(51) Int. Cl. A61F 5/00

(52) U.S. Cl. 602/32

(57) ABSTRACT

A two piece hip distraction system made up of 1) a device for securing a persons leg so traction can be applied to hip and 2) a device for securing a persons torso so traction can be specific to just the femoroacetabular joint. This hip distraction system can be adjusted so various sized people can quickly enter the device with their hip position in the open pack position. A method for distracting a person's hip comprising of steps for securing a person leg and torso to hip distraction system and steps to maintain a distracting force on a person's hip. Both the system and method can accomplish rehabilitative or therapeutic benefit to a dysfunctioned hip.

### FIELD OF INVENTION

The present invention related to rehabilitative and therapeutic devices and more particularly an apparatus for sustaining a constant and variable distraction on a human's hip.

### BACKGROUND OF INVENTION

Arthritis of the hip is a common ailment that affects 40% of the US population over the age of 40. This condition can be severely debilitating and in many cases this condition can progress to the point where costly hip replacements must be done in order to regain some quality of life. The initial signs and symptoms of hip arthritis include pain, stiffness and loss of motion in the hip. In order to minimize these symptoms, it is essential to continually stretch the joint, and exercise the muscles around hip. Currently, medication, physical therapy and chiropractic adjustments are the only way to improve hip function. However, side effects to medication, expensive treatment costs and treatment discomforts make it difficult to maintain patient compliance. A manual technique called hip distraction is a comfortable and affordable way to improve a the functionality of a person's hip. It is performed by placing a hip in the open pack position and sustaining a slow gradual pull away from the acetabulum. This benefits the joint by passively stretching the muscles tendons and ligaments. In addition, hip distraction can allow fluid to circulate within the joint, increasing cell regeneration and joint support. To date, there are no practical devices to accomplishing this rehabilitative technique. Although several medical devices have been developed to traction a hip, these devices are only used in surgical or traumatic environments and none of them can practically be used for rehabilitative or therapeutic means.

For example, U.S. Pat. No. 4,621,625 discloses a device consisting of a leg sling which keeps the ankle and knee in a flexed position while a pulling force is directed away from the persons torso. However, because the leg is not adequately secured to this sling, the sling may slide off as increased force is applied to the leg. This device can apply a distracting force to the femoroacetabular joint, but this joint position is not in an open pack position and therefore hip distraction cannot be maximized. In addition, this device has no body harness to secure the persons torso while hip distraction is taking place, therefore, the applied pulling force is not as specific to the femoroacetabular joint. This device has the ability to control the tension of the pulling force, however it does not allow the person who is positioned in the distraction device to control this pulling force.

U.S. Pat. No. 6,394,972 discloses a device, that immobilizes the ankle and knee and keeps the knee in an fully extended position. A distracting force can be applied, specific and controlled to the hip. However, this device has no means to measure the amount of distracting force applied to the leg. In addition, there is no means of positioning the hip in an open pack position with this device.

Other devices known to applicant include U.S. Pat. No. 5,162,039 which discloses a distraction and reduction device for proper alignment of bone fragments in a fractured femur, and U.S. Pat. No. 5,608,934 which discloses a hip distractor device for positioning a leg and hip during surgery.

### BRIEF DESCRIPTION OF THE DRAWINGS

FIG. 1 is a perspective view of the hip positioning device of the present invention.

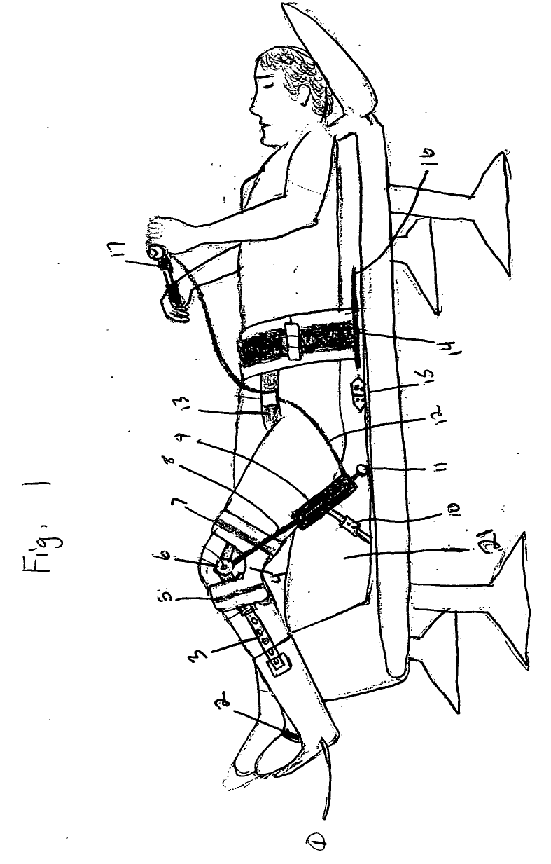

FIG. 2 is a top plan view of the hip positioning device of the present invention.

FIG. 3 is a top plan view of the leg positioning device which forms a part of the invention.

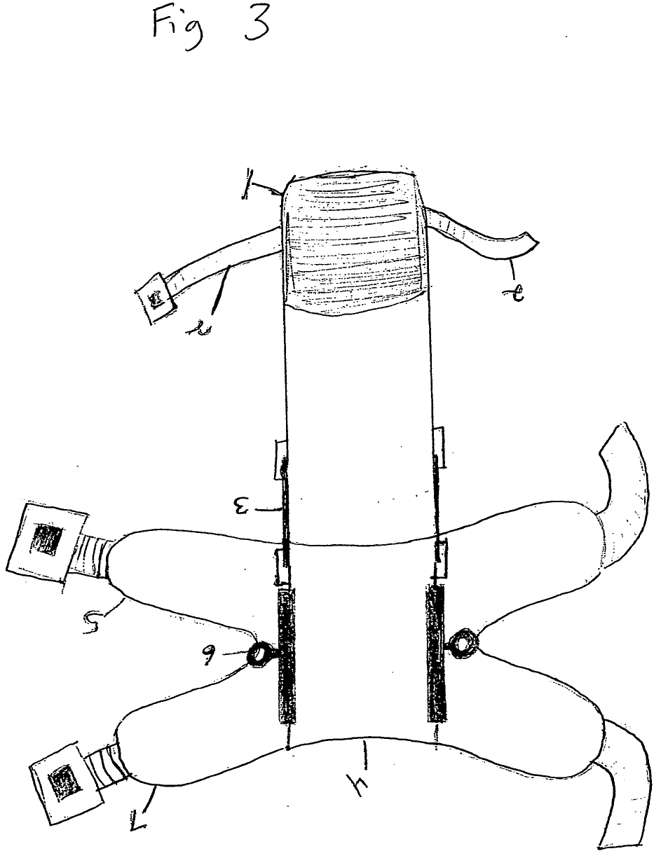

FIG. 4 is a perspective view of the leg positioning device showing an adjustable two piece unit.

FIG. 5 is a top view of the restraining device to restrain a person's torso during hip distraction.

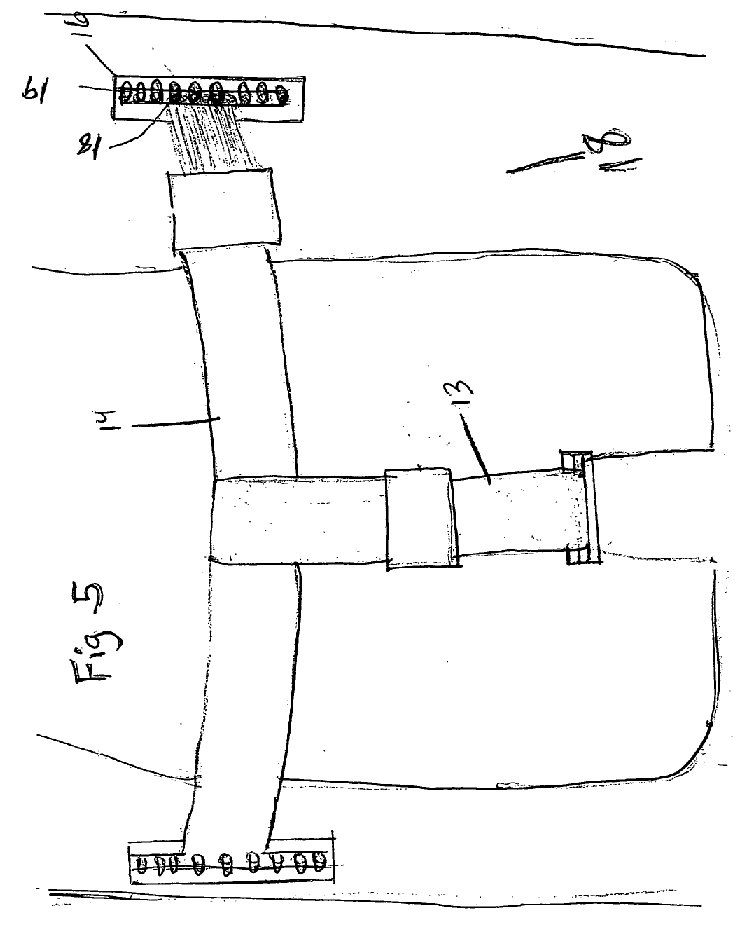

FIG. 6 is a perspective view of the fluid cylinder used to apply a distracting force to the hip.

FIG. 7 is a to view of the securing device for the restraining device which shows how a two ends of a strap can be adjusted relative to their attachments.

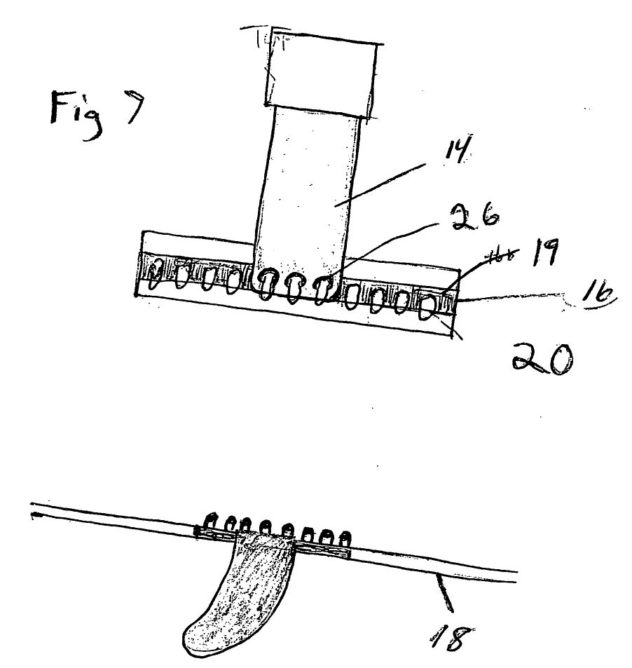

FIG. 8 is a perspective view of a hip positioning device with a reinforced rigid support belt 17.

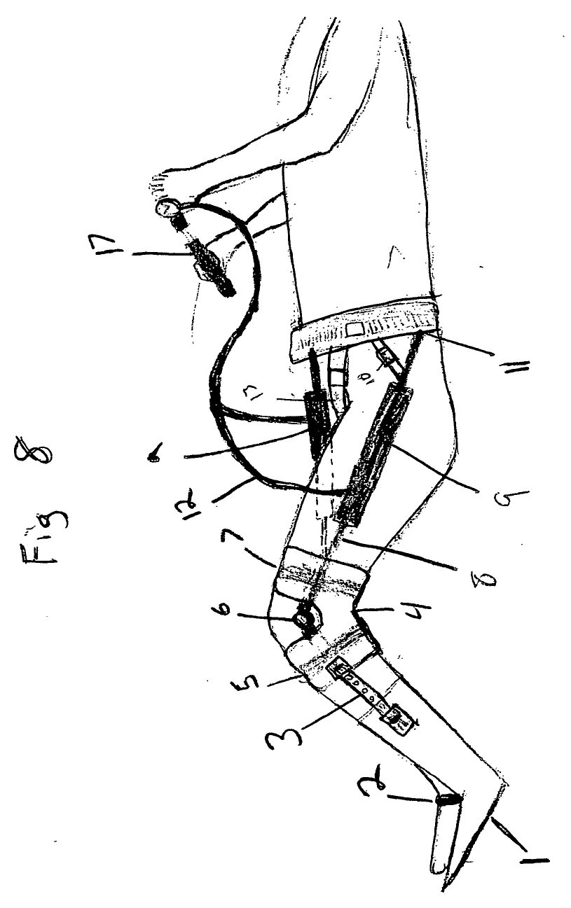

### DETAILED DESCRIPTION OF THE INVENTION

Referring to the drawings, a system for distracting a hip positioning device is indicated generally by the numeral 1. This system 1 provides a means for placing traction on a persons hip and furthermore this system can be utilized for rehabilitative or therapeutic benefits in a dysfunctional hip.

A leg securing device is indicated generally by numeral 3. This system provides a means for securing a persons leg while a force is applied to the persons hip. This device is designed to be applied to a persons leg quickly and efficiently while accommodating various sized legs.

A torso securing device is indicated by numeral 5. This device can secure a persons torso while hip distraction is taking place. This will allow hip distraction to be specific to just the femoralacetabular joint. A It includes a rigid ankle splint 1 fixed to a rigid knee splint 4. The ankle splint places a person's ankle in a dorsiflexed position and is secured by an ankle strap 2 and a shin strap 2a. This ankle splint 1 can be adjusted relative to the knee splint 4 by a sliding support bar 3 between them. This can allow the system 0 to accommodate legs of varying lengths. This support bar 3 slides between the ankle splint 1 and the knee splint 4. A guide shaft 4b on the proximal end of the ankle splint 1 and distal end of the knee splint 5 allow the support bar to slide in a longitudinal direction. When the adjustment is made, the ankle splint 1 and the knee splint 4 are locked to the support bar 3 by tightening a wing nut 4a. A person's knee can be secured to the knee splint 4 by two a subpatellar strap 5 and a suprapatellar strap 7. The Placement of these straps will allow the patient, doctor or therapist to easily and quickly enter the hip distraction system 0. Releasable clamp 6 is fixed to the knee splint 4 and is used to attach a pulling or pushing force.

Fixed to the knee splint 4 is an extending arm 8 which is part of the fluid piston 9. This extending arm can detach from the knee splint 4 by releasing a clamp 6. The fluid piston 8, can attach to either a support board 18 or a rigid body harness 18a. This device can permit the fluid piston to fold up on itself for easy storage. The fluid piston 8 has a collapsible arm 10 which when can allow the fluid piston to either lock in place at a certain angle or lay flat against a support board 18. A releasable swivel 11 can allow the fluid piston 9 to freely move up or down relative to a support board 18. This movement is accomplished by penetrating a cylindrical pin 11b through both a rigid housing 11a and the fluid piston 9. The swivel 11 fixes the fluid piston 9 a predetermined vertical and longitudinal angle relative to the support board 18. This will allow a persons hip to be placed in a fixed abducted and externally rotated position. A conduit tube 12 attached to the fluid piston 9 on one end and a pressure gauge 17 on the other will allow a controlled pressure to be applied to the extending arm 8. This pressure applied to the extending arm 8 will provide a force directly away from a person's femoralacetabular joint.

A person is restrained to the support board 18 by a waist strap 14 and a groin strap 13. These straps will keep a person from moving in the direction of the applied distracting force. A waist buckle 14a allows the waist strap 14 to release into two parts and adjust for various diameters. The groin strap is fixed to the support board 18 and the waist strap 14 and can adjust to various diameters by a groin buckle 13a.

The waist strap can be adjusted up or down the longitudinal axis of the support board by a hook and loop device 16. Gromets 16a are placed at the ends of the waist strap 14 and are sized to fit hooks 16c that are fixed to the support board 18. The hooks 16c are evenly spaced apart and extend linearly along the support board 18 for a span suitable to fit all heights of people.

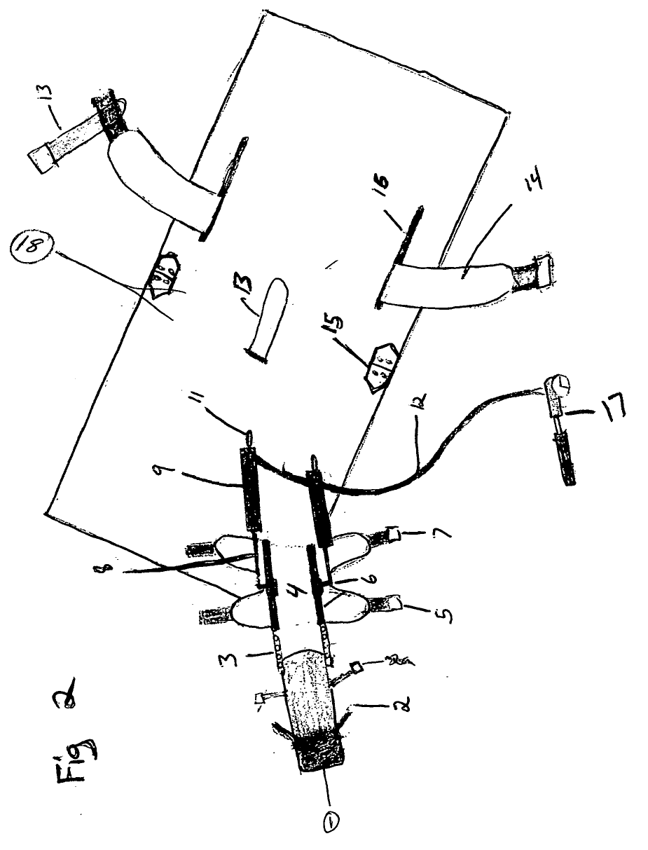

Referring to FIG. 2, two fluid pistons 9 are used to position a person's leg away from the center of the support board 18. This will fix a persons hip in an abducted position. Hinges 15 are fixed on either side of the support board 18 and placed half way up the length of the support board. This will allow the support board to fold in half inwardly for easy storage.

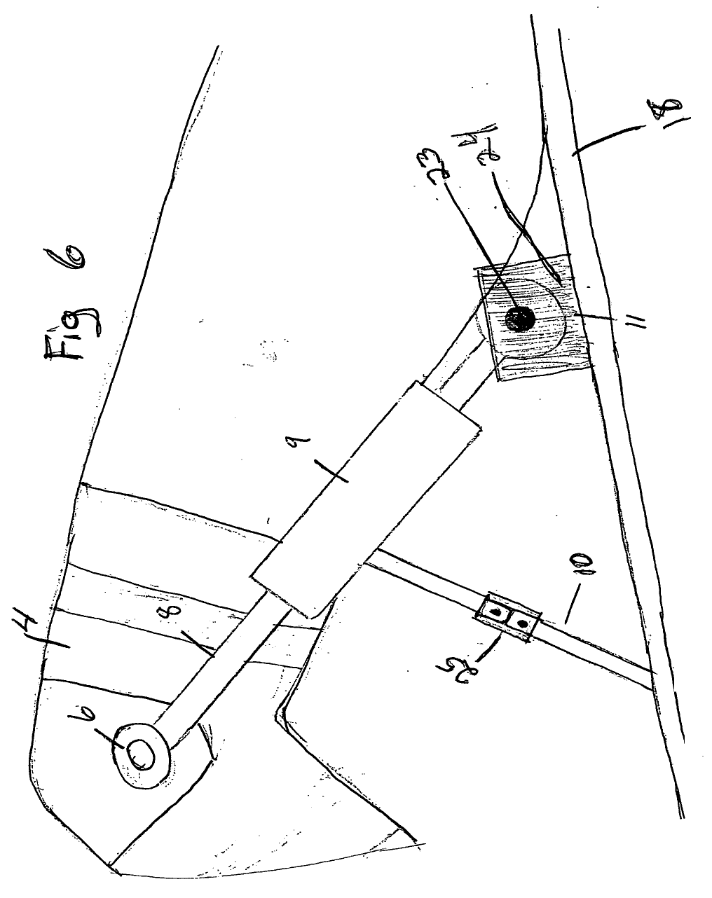

Referring to FIG. 6, a locking hinge 10a allows the collapsible arm 10 to fold in half when fully collapsed and locked when fully extended. The releasable clamp 6 can be used to attach the extending arm 8 of the piston 8. Similarly a pulling force can be applied to this releasable clamp 6 in a similar fashion.

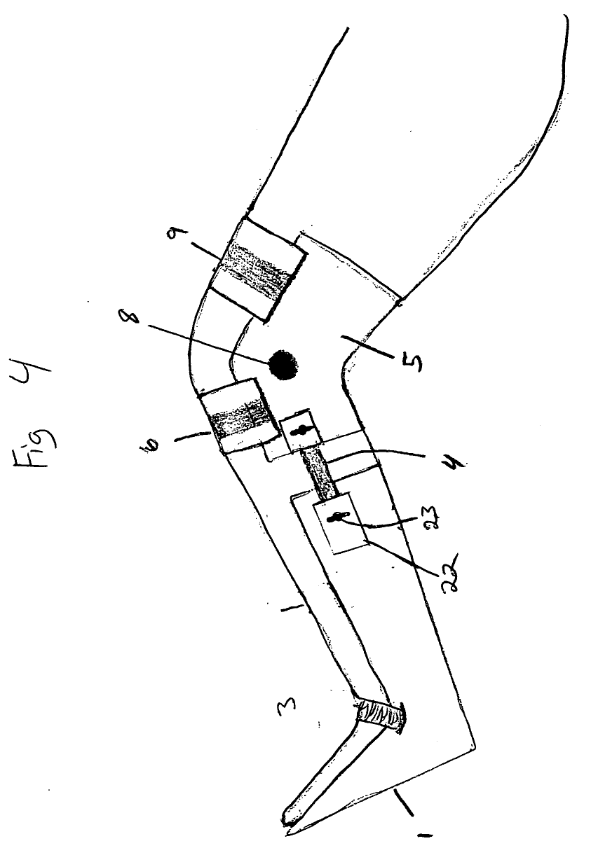

Referring to FIG. 4, the groin strap 13 is looped around the waist strap 14. This will allow the groin strap 13 to be positioned at the center of the waist strap 14 during its size adjustment.

1. A device for positioning a leg of a human, said device comprising:
first positioning means for positioning a knee in a first flexed position;
second positioning means for positioning an ankle in a second dorsiflexed position, adjustment means for adjusting said first positioning means relative to said second positioning means to accommodate legs of varying lengths;
first securing means for securing said first positioning means to said knee; and
second securing means for securing said second positioning means to said ankle;
wherein said first positioning means and said second positioning means are dimensioned and arranged such that positioning and securing said knee in said first flexed position and said ankle in said second dorsiflexed position results in a hip of the human being positioned in an open pack position when the human lies with a back flat on a surface.

2. The device of claim 1 wherein said first positioning means and said second positioning means each comprise a substantially rigid shell and wherein said first securing means and said second securing means comprises hook and loop fasteners.

3. The device of claim 1 wherein said adjustment means comprises at least one support bar connecting said first positing means to said second positioning means.

4. The device of claim 3, wherein said at least one support bar is fixedly attached to one of said first positioning means and said second positioning means, and wherein said adjustment means further comprises a comprises at least one support guide attached to another of said first positioning means and said second positioning means, wherein aid at least one support guide is dimensioned to accept said support bar and allow said support bar to slide within said guide, and wherein said guide comprises means for securing said support bar.

5. The device of claim 4, wherein said means for securing said support bar comprises a releasable clamp.

6. A system for distracting a hip of a human, said system comprising:
a support surface dimensioned to support at least a back and torso of the human;
a device for positioning a leg of a human, wherein said device comprises:
first positioning means for positioning a knee in a first flexed position;
second positioning means for positioning an ankle in a second dorsiflexed position,
adjustment means for adjusting said first positioning means relative to said second positioning means to accommodate legs of varying lengths;
first securing means for securing said first positioning means to said knee; and
second securing means for securing said second positioning means to said ankle;
wherein said first positioning means and said second positioning means are dimensioned and arranged such that positioning and securing said knee in said first flexed position and said ankle in said second dorsiflexed position results in said hip being positioned in an open pack position when the back of the human is disposed upon said support surface;
force applying means for exerting a force upon said device for positioning the leg, wherein said force is directed parallel to a femur of the human and away from an acetabulum of the human when the hip is in the open pack position; and
restraining means for restraining movement of the back and torso of the human relative to the support surface while the hip is distracted.

7. The system of claim 6, wherein said first positioning means and said second positioning means of said device for positioning the leg each comprise a substantially rigid shell and wherein said first securing means and said second securing means comprises hook and loop fasteners.

8. The system of claim 6 wherein said adjustment means of said device for positioning the leg comprises at least one support bar connecting said first positing means to said second positioning means.

9. The system of claim 8, wherein said at least one support bar is fixedly attached to one of said first positioning means and said second positioning means, and wherein said adjustment means further comprises a comprises at least one support guide attached to another of said first positioning means and said second positioning means, wherein aid at least one support guide is dimensioned to accept said support bar and allow said support bar to slide within said guide, and wherein said guide comprises means for securing said support bar.

10. The system of claim 9, wherein said means for securing said support bar comprises a releasable clamp.

11. The system of claim 6 wherein said restraining means comprises a body harness attached to said support surface.

12. The system of claim 11, wherein said support surface comprises a plurality of hooks extending therefrom, and wherein said body harness comprises an adjustable and releasable belt comprising a plurality of grommets dimensioned and disposed to engage said plurality of hooks.

13. The system of claim 12, wherein said body harness further comprises a groin strap.

14. The system of claim 11 wherein said force applying means for exerting a force upon said device for positioning the leg comprises at least one fluid cylinder attached to said first positioning means of said device for positioning the leg and to said support surface.

15. The system of claim 14, further comprising a remote fluid control system for remotely controlling an operation of said at least one fluid cylinder.

16. The system of claim 6 wherein said restraining means comprises a harness disposed about the torso of the human and wherein said force applying means for exerting a force upon said device for positioning the leg comprises at least one fluid cylinder attached to said first positioning means of said device for positioning the leg and to said harness.

17. The system of claim 16, further comprising a remote fluid control system for remotely controlling an operation of said at least one fluid cylinder.

18. The system of claim 11, wherein said support surface comprises folding means for folding said support surface for transport and storage.

19. A method for distracting a hip of a human comprising the steps of:
positioning and securing a knee in said first flexed position and an ankle in a second dorsiflexed position such that said hip is positioned in an open pack position;
restraining movement of a torso of the human; and
exerting a force upon a leg of the human, wherein said force is directed parallel to a femur of the human and away from an acetabulum of the human when the hip is in said open pack position.

20. The method of claim 19 further comprising of step of maintaining said force upon the leg of the human for a period of time between five minutes and twenty minutes.

21. The method of claim 19 wherein said exerting step comprises the step of actuating a fluid cylinder.
